(19) United States
(12) Patent Application Publication
(10) Pub. No.: US 2008/0051648 A1
(43) Pub. Date: Feb. 28, 2008

(54) MEDICAL IMAGE ENHANCEMENT SYSTEM

(76) Inventors: Jasjit S. Suri, Roseville, CA (US); Dinesh Kumar, Grass Valley, CA (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/609,743

(22) Filed: Dec. 12, 2006

Related U.S. Application Data
(60) Provisional application No. 60/823,536, filed on Aug. 25, 2006.

(51) Int. Cl. A61B 5/05

(52) U.S. Cl. 600/407

(57) ABSTRACT

Provided herein is a medical imaging system that allows for real-time guidance of, for example, catheters for use in interventional procedures. In one arrangement, an imaging system is provided that generate a series of images or frames during a dye injection procedure. The system is operative to automatically detect frames that include dye (bolus frames) and frames that are free of dye (mask frames). The series of images may be registered together to provide a common reference frame and thereby account for motion. Sets of mask frames and bolus frames are averaged together, respectively, to improve signal to noise qualities. A differential image is generated utilizing the average mask and average bolus frames. Contrast of the differential image may be enhanced. The system allows for motion correction, noise reduction and/or enhancement of a differential image in real time.

### CROSS-REFERENCE TO RELATED APPLICATION

This application claims priority under 35 U.S.C. §119 to U.S. Provisional Application No. 60/823,536 having a filing date of Aug. 26, 2006, the entire contents of which are incorporated by reference herein.

### FIELD

The present disclosure is directed to medical imaging systems. More specifically, the present disclosure is directed to systems and methods that alone or collectively facilitate real-time imaging.

### BACKGROUND

Interventional medicine involves the use of image guidance methods to gain access to the interior of deep tissue, organs and organ systems. Through a number of techniques, interventional radiologists can treat certain conditions through the skin (percutaneously) that might otherwise require surgery. The technology includes the use of balloons, catheters, microcatheters, stents, therapeutic embolization (deliberately clogging up a blood vessel), and more. The specialty of interventional radiology overlaps with other surgical arenas, including interventional cardiology, vascular surgery, endoscopy, laparoscopy, and other minimally invasive techniques, such as biopsies. Specialists performing interventional radiology procedures today include not only radiologists but also other types of doctors, such as general surgeons, vascular surgeons, cardiologists, gastroenterologists, gynecologists, and urologists.

Image guidance methods often include the use of an X-ray picture (e.g., a CT scan) that is taken to visualize the inner opening of blood filled structures, including arteries, veins and the heart chambers. The X-ray film or image of the blood vessels is called an angiograph, or more commonly, an angiogram.

Angiograms require the insertion of a catheter into a peripheral artery, e.g. the femoral artery. The tip of the catheter is positioned either in the heart or at the beginning of the arteries supplying the heart, and a special fluid (called a contrast medium or dye) is injected.

As blood has the same radiodensity as the surrounding tissues, the contrast medium (i.e. a radiocontrast agent which absorbs X-rays) is added to the blood to make angiography visualization possible. The angiographic X-Ray image is actually a shadow picture of the openings within the cardiovascular structures carrying blood (actually the radiocontrast agent within). The blood vessels or heart chambers themselves remain largely to totally invisible on the X-Ray image. However, dense tissue (e.g., bone) are present in the X-Ray image and are considered what is termed background.

The X-ray images may be taken as either still images, displayed on a fluoroscope or film, useful for mapping an area. Alternatively, they may be motion images, usually taken at 30 frames per second, which also show the speed of blood (actually the speed of radiocontrast within the blood) traveling within the blood vessel.

### SUMMARY

It is sometimes possible to remove background (i.e., structure such as dense tissue and bones) from an image in order to enhance the cardiovascular structures carrying blood. For instance, an image taken prior to the introduction of the contrast media and an image taken after the introduction of contrast media may be combined (e.g., subtracted) to produce an image where background is significantly reduced. In this regard, the images after dye injection (also referred to as bolus images) contain background structure as well as the cardiovascular structure as represented by the contrast media therein. In contrast, the images before dye injection (also referred to as mask images) contain only background. If there is no patient movement during the image acquisition, the difference between the images (e.g., subtraction of these images) should remove the background and the image regions enhanced by the contrast media (i.e., blood vessels) should remain in the difference image.

However, movement occurring between acquisition of the mask and bolus images complicates this process. For example, patient breathing, heartbeat and even minor movement/shifting of a patient result in successive images being offset. Stated otherwise, motion artifacts exist between different images. Accordingly, simply subtracting a mask image from a bolus image (or vice versa) can result in blurred images. One response to this problem has been to select a mask image and bolus image that are as temporally close as possible. For instance, the last mask image prior to the infiltration of contrast media into the images may be selected as the mask image. Likewise, the first bolus image where contrast media is visible or where contrast media is visible and reached a steady state condition (e.g., spread throughout the image) may be selected as the bolus image. However, such selection has previously required manual review of the images to identify the mask and bolus images. Such a process has not been useful for real-time image and guidance systems.

The inventors have recognized that in various imaging systems (e.g., CT, fluoroscopy etc) images are acquired at different time instants and generally consist of a movie with a series of frames (i.e., images) before, during and after dye injection. Frames are therefore, available for mask images that are free of dye in their field of view and bolus images having contrast-enhancing dye in their field of view. Further, it has been recognized that it is important to detect the frames before and after dye injection automatically to make a real-time imaging and guidance system possible. One approach for automatic detection is to find intensity differences between successive frames, such that a large intensity difference is detected between the first frame after dye has reached the field of view (FOV) and the frame acquired before it. However, the patient may undergo some motion during the image acquisition causing such an intensity differences exist between even successive mask images.

One method for avoiding this is to align successive frames together such that the motion artifacts between successive frames are minimized. For instance, image registration of successive images may provide a point-wise correspondence between successive images such that these images share a common frame of reference. That is, successive frames are motion corrected such that a subtraction or differential image obtained after motion correction will contain a near-zero value everywhere if both images are free of dye in their field of view (i.e., are mask frames). The first image acquired after the dye has reached the field of view will therefore cause a high intensity difference with the previous frame not containing the dye in field of view. Accordingly, detection of such an intensity difference allows for the automated detection of the temporal reference point between mask frames free of dye and bolus frames containing dye. Likewise, a mask frame before the reference point and a bolus frame after the reference point may be selected to generate a differential image.

It has also been determined that it may be beneficial to compute an average of a set of mask frames and an average of the bolus frames rather than using one of each of the frames for computing the difference image. For instance, the previous four registered frames (e.g., registered to share a common reference frame) may be collected as the mask frames, and the consecutive four registered bolus frames with dye in the field of view may be collected as the bolus frames. The four bolus frames and four mask frames may be averaged together to reduce noise and slight registration errors.

The average mask and average bolus frames may still contain motion artifacts, since these frames are temporally spaced apart. Accordingly, these average images may be registered together to account for such motion artifact (i.e., place the images in same frame of reference). An inverse-consistent intensity based image registration may be used to align the bolus image to the mask image. The method minimizes the symmetric squared intensity differences between the images and registers the bolus into co-ordinate system of the average mask frame. A subtraction process is performed between the registered bolus frame and the average mask frame to produce a differential image. This is called a “DSA image”. The DSA image is substantially free of motion artifact due to breathing and is also substantially free from any artifacts such as catheter movement or deformation of the blood vessel anatomy by the pressure of the catheter.

However, the image may still contain some noise that may be caused by, for example, system noise caused by the imaging electronics. For instance, the images may contain dotty patterns (salt-and-pepper noise). Accordingly, the DSA image may be de-noised before performing additional enhancement. In one arrangement, the noise characteristics of the image are improved using a method based on scale-structuring such as wavelet based method or a diffusion based noise removal.

The motion free DSA image may then be enhanced using different methods that may be based on classification of pixels into foreground and background pixels. The foreground pixels are typically the pixels in the blood vessels, while the background pixels are typically non-blood vessel pixels are tissue pixels. One enhancement method classifies the image into foreground and background regions and weights differently depending upon the foreground and background pixels. This weighing scheme uses strategy where the weights are distributed in a non-linear framework at every pixel location in image. A second method divides the image into more than two classes to better tune the non-linear enhancement into a more structured method, which is represented into piece-wise form.

The method is very robust and shows the drastic improvement in image enhancement methodology while allowing for real-time motion correction of a series of images, identification of dye infiltration, generation of a differential image and de-noising and enhancement of the differential image. Accordingly, the method, as well as novel sub-components of the method allow for real-time imaging and guidance. That is, the resulting differential image may be displayed for real time use.

According to a further aspect, a system and method (i.e., utility) for use in a real-time medical imaging system is provided. The utility includes obtaining a plurality of successive images having a common field of view, the images being obtained during a contrast media injection procedure. A first set of the plurality of images is identified that are free of contrast media in their field of view. A second set of the plurality of images is identified that contain contrast media in the field of view. A differential image is then generated that is based on a first composite image associated with the first set of images and a second composite image associated with the second set of images. This differential may then be displayed on a user display such that the user may guide a medical instrument based on the display.

The first and second sets of images may be identified in automated process such that the differential image may be generated in real-time. The automated process includes computing intensity differences between temporally adjacent images and identifying the intensity difference between two temporally adjacent images where the intensity difference is indicative of contrast media being introduced into the latter of the two adjacent images. Such identification of the two adjacent images where the first image is free of dye and the second image contains dye within the field of view may define a contrast media introduction reference time. The first set of images may be selected before the reference time, and the second set of images may be selected after the reference time.

In the first arrangement, each successive image may be registered to the immediately preceding image. In this regard, each of the images may share a common frame of reference. In one arrangement, the images are registered utilizing a bi-directional registration method. Such a bi-directional registration method may include use of an inverse consistent registration method. Such a registration method may be computed using a B-spline parameterization. Such a process may reduce computational requirements steps and thereby facilitate the registration process being performed in substantially real-time.

In a further arrangement, the differential image may be further processed to enhance the contrast between the contrast media, as represented in the differential image, and background information, as represented in the differential image. Such enhancement may entail resealing the pixel intensities of the differential image. In one arrangement, this resealing of pixel intensities is performed in a linear process based on the minimum and maximum intensity values of the differential image. For instance, the minimum and maximum intensity differences and all intensities in between may be resealed to a full range (e.g., 1 thru 255) to allow for improved contrast. In a further arrangement, a subset of the differential image may be selected for enhancement. For instance, a region of interest within the image may be selected for further enhancement. In this regard, it is noted that the edges of many images often contain lower intensities. By eliminating such low intensity areas, the intensity difference in the region of interest (i.e., the difference between the minimum and maximum intensity values) may be reduced. Accordingly, by redistributing these intensities over a full intensity range, increased enhancement may be obtained.

In another arrangement, enhancing the contrast includes performing a nonlinear normalization to rescale the pixel intensities of the differential image. Such nonlinear normalization may be performed in first and second pixel intensity bands. In further arrangements, nonlinear normalization may be performed in a plurality of pixel intensity bands.

In a further aspect, a utility is provided for use in a real-time medical imaging system. The utility includes obtaining a plurality of successive images having a common field of view where the images are obtained during a contrast media injection procedure. Each of the plurality of images may be registered with a temporally adjacent image to generate a plurality of successive registered images. The intensities of temporally adjacent registered images may be compared to identify a first image where contrast media is visible. For instance, identifying may include identifying an intensity difference between adjacent images that is greater than a predetermined threshold and thereby indicative of dye being introduced into the subsequent image.

In another aspect, a utility for use in a real-time medical imaging system is provided. The utility includes obtaining a plurality of successive images having a common field of view where the images are obtained during a contrast media injection procedure. Each of the plurality of images may be registered at temporally adjacent images to generate a plurality of registered images. A first set of mask images that are free of contrast media may be averaged to generate an average mask image. Likewise, a set of bolus images containing contrast media in their field of view may be averaged to generate an average bolus image. A differential image may be generated based on differences between the average mask image and the average bolus image. In further arrangements, de-noising processes may be performed on the differential image to reduce system noise. Further, intensities of the differential image may be enhanced utilizing, for example, linear and nonlinear enhancement processes.

### BRIEF DESCRIPTION OF THE DRAWINGS

FIG. 1 illustrates one embodiment of the system.

FIG. 2 illustrates a process flow diagram of in interventional procedure.

FIG. 3 illustrates further process flow diagram of the interventional procedure of FIG. 2.

FIG. 4 illustrates a process flow diagram of the X-ray movie acquisition system with enhancement.

FIG. 5 illustrates a process flow diagram of the process of movie enhancement.

FIG. 6 illustrates process flow diagram for the mask frame identification.

FIG. 7 illustrates a process flow diagram of registration for mask identification.

FIG. 8 illustrates a process flow diagram of frame alignment for mask identification.

FIG. 9 illustrates a process flow diagram for a image registration system.

FIG. 10 illustrates a process flow diagram for gradient cost computation for registration.

FIG. 11 illustrates a process flow diagram for updating deformation parameters for an image registration system.

FIG. 12 illustrates a process flow diagram for producing an DSA image including noise reduction and enhancement.

FIG. 13 illustrates a process flow diagram of a DSA generation system.

FIG. 14 illustrates a process flow diagram of a mask averaging system.

FIG. 15 illustrates a process flow diagram of a bolus averaging system.

FIG. 16A illustrates process flow diagram for noise removal for a DSA image.

FIG. 16B illustrates an edge band removal process for normalization.

FIG. 17 illustrates a process flow diagram for a LUT enhanced DSA system.

FIG. 18 illustrates a process flow diagram for the 3-Class LUT enhanced DSA system.

### DETAILED DESCRIPTION

Reference will now be made to the accompanying drawings, which assist in illustrating the various pertinent features of the various novel aspects of the present disclosure. Although the present invention will now be described primarily in conjunction with angiography utilizing X-ray imaging, it should be expressly understood that aspects of the present invention may be applicable to other medical imaging applications. For instance, angiography may be performed using a number of different medical imaging modalities, including biplane X-ray/DSA, magnetic resonance (MR), computed tomography (CT), ultrasound, and various combinations of these techniques. In this regard, the following description is presented for purposes of illustration and description. Furthermore, the description is not intended to limit the invention to the form disclosed herein. Consequently, variations and modifications commensurate with the following teachings, and skill and knowledge of the relevant art, are within the scope of the present invention. The embodiments described herein are further intended to explain known modes of practicing the invention and to enable others skilled in the art to utilize the invention in such, or other embodiments and with various modifications required by the particular application(s) or use(s) of the present invention.

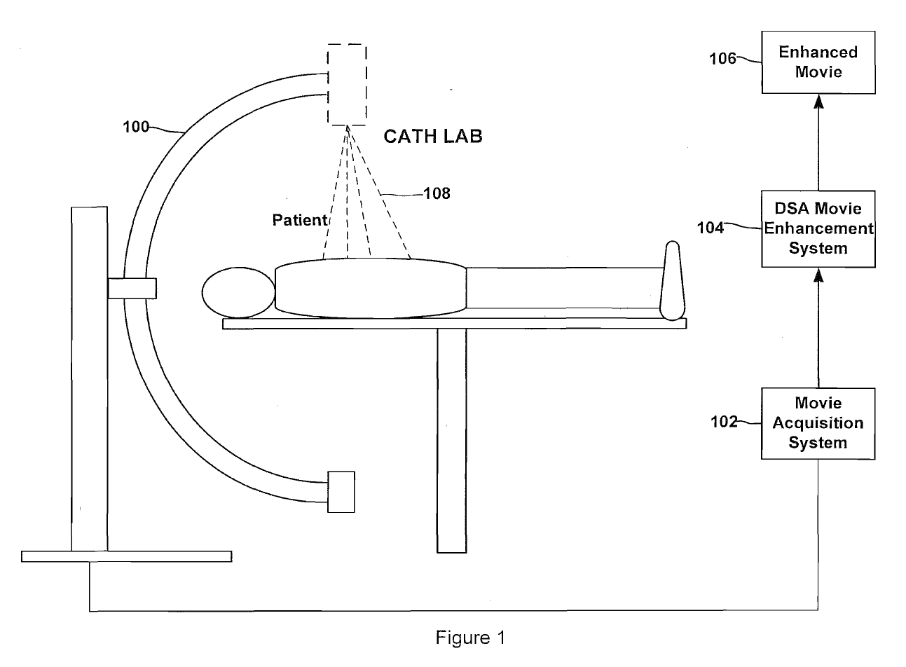

FIG. 1 shows one exemplary setup for a real-time imaging procedure for use during a contrast media/dye injection procedure. As shown, a patient is positioned on an X-ray imaging system 100 and an X-ray movie is acquired by a movie acquisition system (102). An enhanced DSA image, as will be more fully discussed herein, is generated by an enhancement system (104) for output to a display (106) that is accessible to (i.e., within view of) an interventional radiologist. The interventional radiologist may then utilize the display to guide a catheter internally within the patient body to a desired location within the field of view of the images.

The projection images (e.g., CT images) are acquired at different time instants and consist of a movie with a series of frames before, during and after the dye injection. The series of frames include mask images that are free of contrast-enhancing dye in their field of view (108) and bolus images that contain contrast-enhancing dye in their field of view (108). That is, bolus frames are images that are acquired after injected dye has reached the field of view (108). The movie acquisition system (102) is operative to detect the frames before and after dye injection automatically to make feasible a real-time acquisition system. As will be discussed herein, one approach for identifying frames before and after dye injection is to find intensity differences between successive frames, such that a large intensity difference is detected between the first frame after dye has reached the field of view (FOV) and the frame acquired before it. However, the patient may undergo some motion during the image acquisition causing such an intensity difference between even successive mask images. To avoid this, the movie acquisition system (102) may align successive frames together, such that the motion artifacts are minimized. The first image acquired after the dye has reached the FOV will therefore cause a high intensity difference with the previous frame not containing the dye in FOV. The subtraction image or ‘DSA image’ obtained by subtracting a mask frame from a bolus frame (or vice versa) will contain a near-zero value everywhere if both images belong to background.

Generally, the subtraction image or DSA image is obtained by computing a difference between pixel intensities of the mask image and the bolus image. The enhancement system (104) may then enhance the contrast of the subtraction image. Such enhancement may include resealing the intensities of the pixels in the subtraction image and/or the removal of noise from the subtraction image. Once enhanced, the resulting real-time movie is displayed (106). These processes are more fully discussed herein.

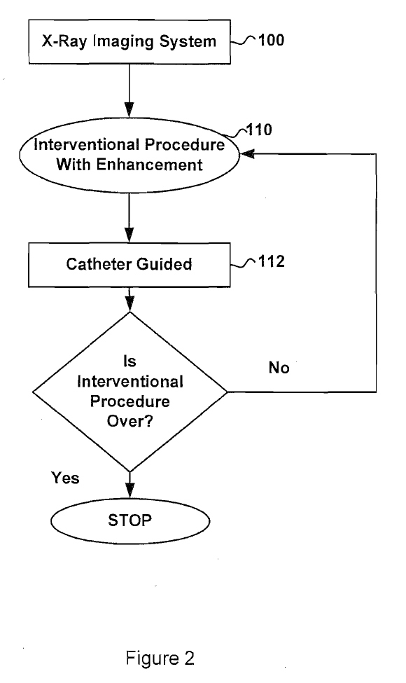

FIG. 2 shows the overall system for the application of presented method in a clinical setup for image-guided therapy. An X-ray imaging system (100) is used to acquire a number of projection images from the patient before during and after dye is injected into patient's blood stream to enhance the contrast of blood vessels (i.e., cardiovascular structure) with respect to background structure (e.g., tissue, bones, etc.). A combined interventional procedure enhancement system (110), which may include the movie acquisition system and enhancement system, produces an enhanced sequence of images of the blood vessels. The enhanced DSA image is used for guiding (112) a catheter during an interventional procedure. The process may be repeated as necessary until the catheter is positioned and/or until interventional procedure is finished.

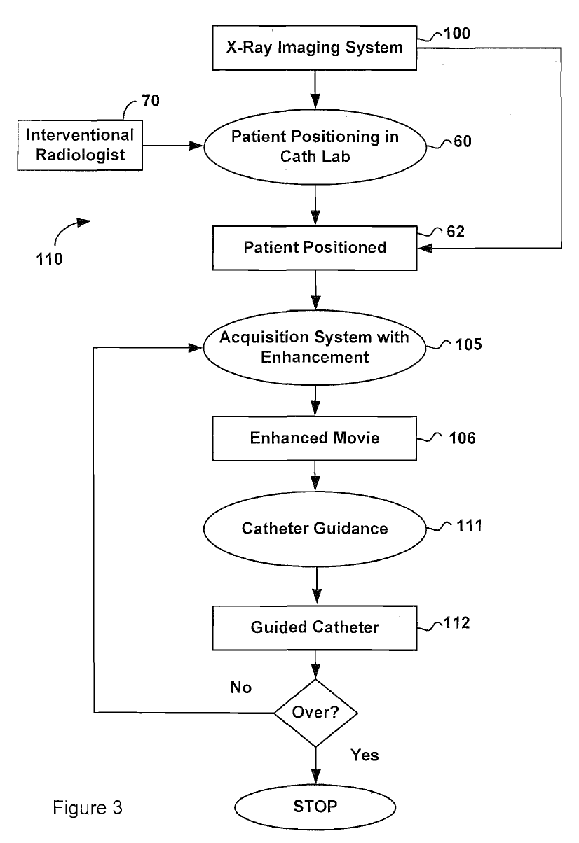

FIG. 3, illustrates one exemplary process flow diagram of an interventional procedure (1 10). Again, an X-ray imaging system (100) is used to acquire a number of projection images from a patient positioned (60) in a catheter lab by, for example an interventional radiologist (70). More specifically, the patient is positioned (60) in the X-ray imaging system (100) such that the area of interest lies in the field of view. Such a process of positioning may be repeated until the patient is properly positioned (62). A sequence of projection images are acquired and enhanced DSA image is created through the acquisition system with enhancement (105), which may include, for example, the movie acquisition system (102) and enhancement system (104) of FIG. 1. The enhanced image sequence is displayed (106) is used for a catheter guidance procedure (111) during the interventional procedure. Such guidance (111) may continue until the catheter is guided (112) one or more target locations where an interventional procedure is to be performed.

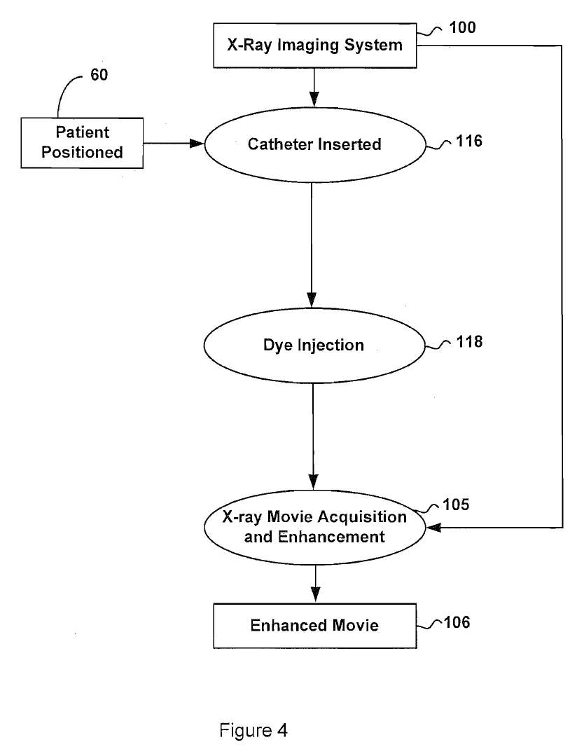

FIG. 4 shows a flowchart of an acquisition system with enhancement. Again, a patient is positioned (60) relative to an X-ray imaging system (100). After inserting (116) the catheter and injection (118) of the dye, the patient X-ray movie acquisition is performed and the movie is enhanced by the for assisting interventional cardiologist. Images are acquired while the patient is given a dye injection (118) with contrast enhancing agent. The X-ray movie is acquired by a combined acquisition and enhancement system (111) and the subtraction/DSA image is created and enhanced in the X-ray by the combined acquisition and enhancement system (111). The acquisition system with enhancement generates an output/display (106) in the form of an enhanced movie for better and clearer visualization of structures.

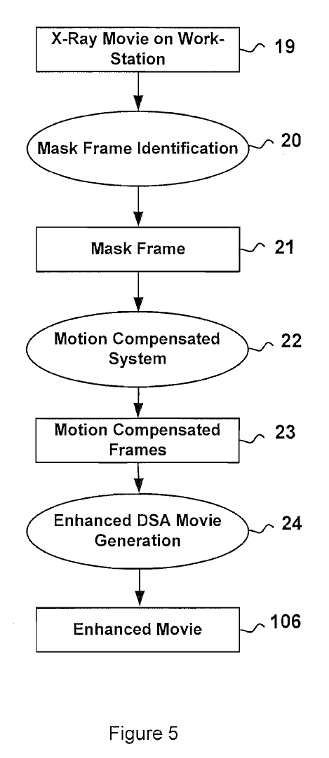

FIG. 5 shows the process through which the acquired image is used to create an enhanced DSA image. On a work station such as the acquisition system (e.g., system 102 of FIG. 1), the mask frames are extracted from the successive frames/images of the obtained X-ray movie. The X-ray movie is transferred to a workstation (19) and one or more mask frames (21) are identified using an automatic mask frame identification method (20). As more fully discussed herein, the mask frame identification method identifies the temporal time where dye first appears. That is, the mask frame identification method identifies a time before which the frames are mask frames (21) and a time after which the frames are bolus frames. The frames (all frames including mask and bolus frames) are motion compensated (22), which is also referred to as registration, to account for patient and internal structural movements and the motion compensated frames are passed through the DSA movie enhancement system. In one arrangement, the acquired frames are aligned together in the process of extracting the mask frames and are motion compensated (22) using a non-rigid inverse consistent image registration method. This produces a series of motion compensates mask and bolus frames (23). As further discussed herein, a set of motion compensated mask frames are averaged together to further reduce motion artifacts. Likewise a set of motion compensated bolus frames are averaged together. The motion compensated average mask and bolus images are then registered together to compute a DSA movie (24) which may then be displayed (106) as discussed above. Of note, the frames/images need to be registered before computing the average image to improve the accuracy of the averages. The images before dye reaches the FOV and after the dye has reached the FOV also need to be registered together for motion compensation. The subtraction image after registration may be enhanced using a linear normalization process, or non-linear or piecewise non-linear intensity normalization process. The steps involved in creating the enhanced movie are discussed below in further detail herein.

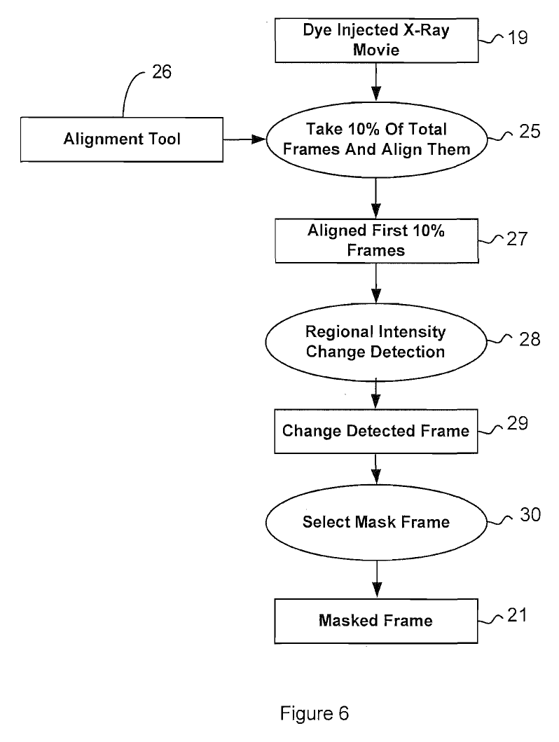

FIG. 6 shows a flow diagram of a procedure used for mask frame identification (e.g., step 20 of FIG. 5). Again, projection image data is available in the form of a number/series of frames acquired at different time instants while the patient is given a contrast enhancement dye injection (19). The collection of frames starts with the field of view containing the structural image before the dye has reached it, and as the dye reaches the field of view. Accordingly, the contrast of blood vessels changes throughout the series of frames. An important task is to pick a set of background structural frames (e.g., 4 mask images) before the dye reaches the field of view and a set of frames after the dye has reached the field of view (e.g., 4bolus images). Previously, this has been performed manually by a human observer, who decides the images to be used as mask and as bolus images, respectively. The presented method incorporates an automatic approach to eliminate the human interaction.

The method is based on the knowledge that the underlying anatomical structure in the field of view remains the same during the mask frames and during the bolus frames. If there is no movement of underlying structure, then the only difference between the first frame containing dye and the previous frame not containing the dye will be in the region containing the dye, i.e. blood vessels. This difference occurs in a cluster at the pixels corresponding to blood vessels. The difference is quite high and can be easily detected. However, in general the image frames are not in same frame of reference and there is some motion of structures in the field of view due to movement of internal anatomical structures and/or the movement of the patient. This causes a high intensity difference even between temporally adjacent frames not containing the dye. This problem is addressed by correcting the adjacent frame for motion using an image registration described herein in next section. As shown in FIG. 6, starting with the first 10% frames, each frame is registered by an alignment module (26) with the adjacent next frame (25). This generates a set of registered or ‘aligned’ frames (27). An intensity difference is calculated (28) for each pair of adjacent frames. After motion-correction using registration, the pixel-wise intensity difference between the successive frames will be very low and almost negligible. However, when first frame with dye in the field of view is reached, the intensity differences will increase by a large amount and can be easily detected (28).

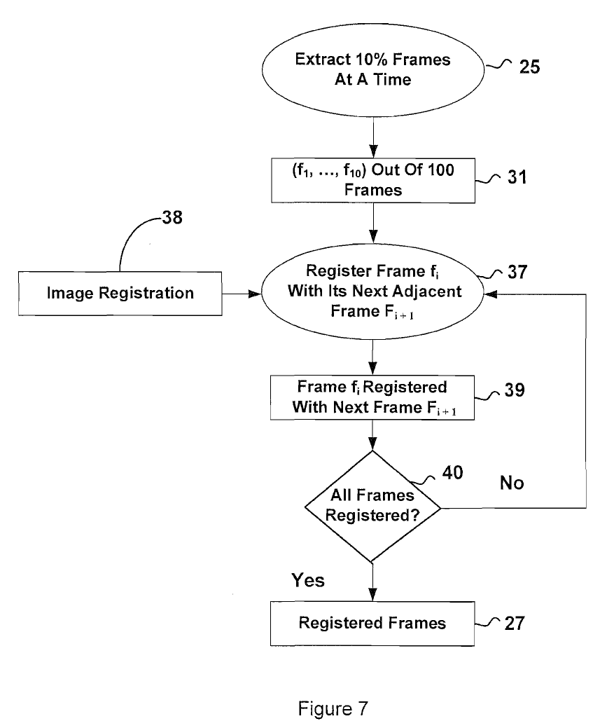

FIG. 7 shows a process flow diagram for motion compensating adjacent frames for mask identification (i.e., step 25 of FIG. 6). As shown, the process registers 10% frames at a time, starting with first 10%. Each frame is registered (37) by an image registration system (38) with next image until all frames are registered with next consecutive image (39,40). The registered frames (27), see FIG. 6, may then be utilized to identify a reference time where images proceeding the reference time are mask images and images subsequent to the reference time are bolus images.

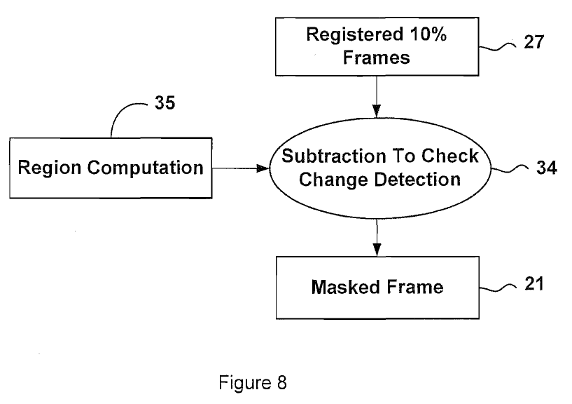

FIG. 8 illustrates process flow diagram where subtraction (34) is performed between adjacent registered frames to detect any large regional changes (e.g., step 28 of FIG. 6). A large regional change between successive frames correspond to an initial ‘masked frame’ where dye has reached the field of view. If intensity difference is detected, i.e. upon detection of masked frame reference point, the four frames before the masked frame reference point are selected (30) as the mask images and the first four frames of images with dye will be used as the bolus images. See FIG. 6. Let n represent the frame number for the first image containing the dye, and let Fₙ represent the image corresponding the frame no. n, then Fₙ₋₄, Fₙ₋₃, Fₙ₋₂ and Fₙ₋₁ are selected as the mask images, while Fₙ, Fₙ₊₁, Fₙ₊₂, Fₙ₊₃ and Fn+4 are selected as the bolus images. Like the mask images, the bolus images are also registered together.

### Image Registration System

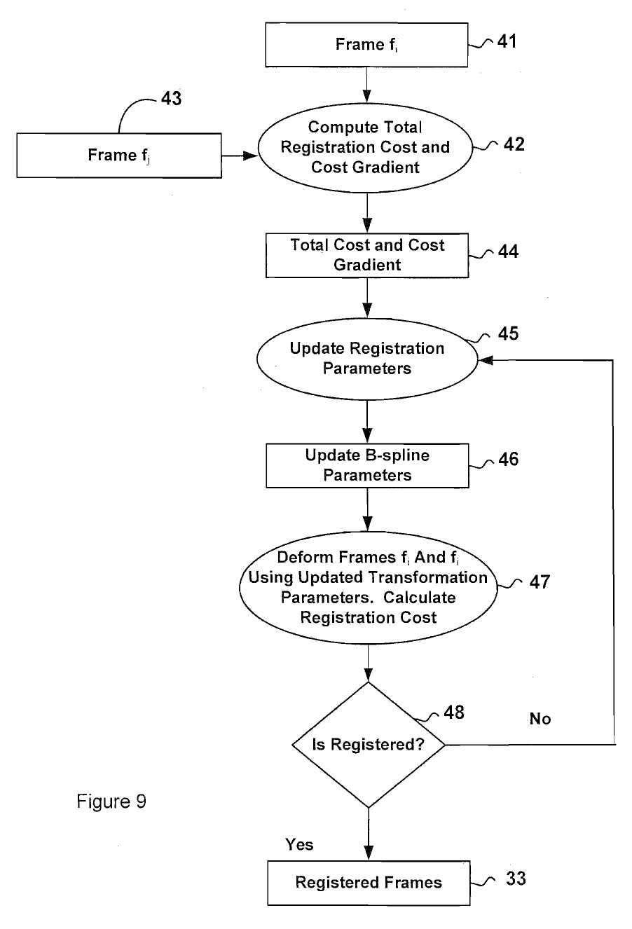

In medical imaging, image registration is performed to find a point-wise correspondence between a pair of images. The purpose of image registration is to establish a common frame of reference for a meaningful comparison between the two images. Image registration is often posed as an optimization problem which minimizes an objective function representing the difference between two images to be registered. FIG. 9 details the image registration system for registering two images together. The registration system takes as input, two images to be registered together (41, 43) using a squared intensity difference as the driving function. This is performed in conjunction with regularization constraints that are applied so that the deformation follows a model that matches closely with the deformation of real-world objects. The regularization is applied in the form of bending energy and inverse-consistency cost. Inverse-consistency implies that the correspondence provided by the registration in one direction matches closely with the correspondence in the opposite direction. Most image registration methods are uni-directional and therefore contain correspondence ambiguities originating from choice of direction of registration. The forward and reverse correspondences are evaluated together and bind them together with an inverse consistency cost term such that a higher cost is assigned to transformations deviating from being inverse-consistent. A cost function of Christensen G. E. Christensen, H. J. Johnson, Consistent Image Registration, IEEE Trans. Medical Imaging, 20(7), 568-582, July 2001, which is incorporated by reference, is utilized for performing image registration over the image:

$$C = σ ( {\int }_{Ω} {\vert {I}_{1} ( {h}_{1 , 2} ( x ) ) - {I}_{2} ( x ) \vert}^{2} d x + {\int }_{Ω} {\vert {I}_{2} ( {h}_{2 , 1} ( x ) ) - {I}_{1} ( x ) \vert}^{2} d x ) + ρ ( {\int }_{Ω} {‖ L ( {u}_{1 , 2} ( x ) ) ‖}^{2} d x + {\int }_{Ω} {‖ L ( {u}_{2 , 1} ( x ) ) ‖}^{2} d x ) + χ ( {\int }_{Ω} {‖ {h}_{1 , 2} ( x ) - {h}_{2 , 1}^{- 1} ( x ) ‖}^{2} d x + {\int }_{Ω} {‖ {h}_{2 , 1} ( x ) - {h}_{1 , 2}^{- 1} ( x ) ‖}^{2} d x ) \qquad ( 1 )$$

where, I₁(x) and I₂(x) represent the intensity of image at location x, represents the domain of the image. h_{i,j}(x)=x+uᵢⱼ(x) represents the transformation from image Iᵢ to image Iⱼ and u(x) represents the displacement field. L is a differential operator and the second term in Eq. (1) represents an energy function. σ, ρ and χ are weights to adjust relative importance of the cost function.

In equation (1), the first term represents the symmetric squared intensity cost function and represents the integration of squared intensity difference between deformed reference image and the target image in both directions. The second term represents the energy regularization cost term and penalizes high derivatives of u(x). In our work, we use L as the Laplacian operator. The last term represents the inverse consistency cost function, which penalizes differences between transformation in one direction and inverse of transformation in opposite direction. The total cost is computed as a first step in registration (42).

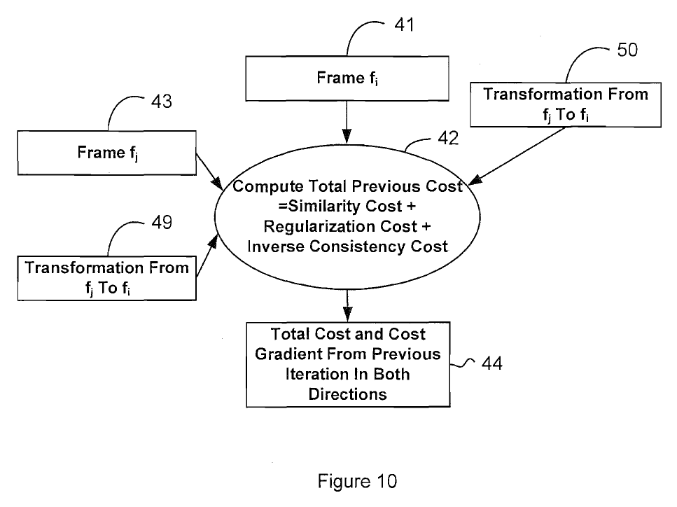

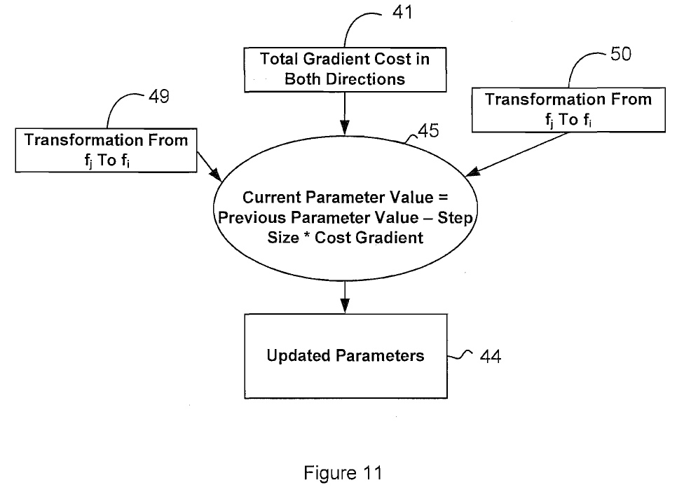

The optimization problem posed In Eq. (1) is solved by using a B-spline parameterization as in the work of Kybic and D. Kumar, X.Geng, Eric A. Hoffman, G. E. Christensen, BICIR: Boundary-constrained inverse consistent image registration using WEB-splines, IEEE conf. Mathematical Methods in Bio-medical Image Analysis, June 2006, which is incorporated by reference and in the work of Kumar and Christensen. B-splines are chosen due to ease of computation, good approximation properties and their local support. It is also easier to incorporate landmarks in the cost term if we use spatial basis function. The above optimization problem is solved by solving for b-spline coefficients cᵢ's, such that

$$h ( x ) = x + {\sum }_{i} {c}_{i} {β}_{i} ( x ) \qquad ( 2 )$$

where, βᵢ(x) represents the value of b-spline at location x, originating at index i. In the registration method, cubic b-splines are used. A gradient descent scheme is implemented based on the above parameterization. The total gradient cost is calculated with respect to the transformation parameters in every iteration (42). The transformation parameters are updated using the gradient descent update rule (FIGS. 10 and 11). Images are deformed into shape of one another using the updated correspondence and the cost function and gradient costs are calculated (47) until convergence (48).

The registration is performed hierarchically using a multi-resolution strategy in both, spatial domain and in domain of basis functions. The registration is performed at ¼^{th},½ and full resolution using knot spacing of 8, 16 and 32. In addition to being faster, the multi-resolution strategy helps in improving the registration by matching global structures at lowest resolution and then matching local structures as the resolution is refined.

### Enhanced DSA System

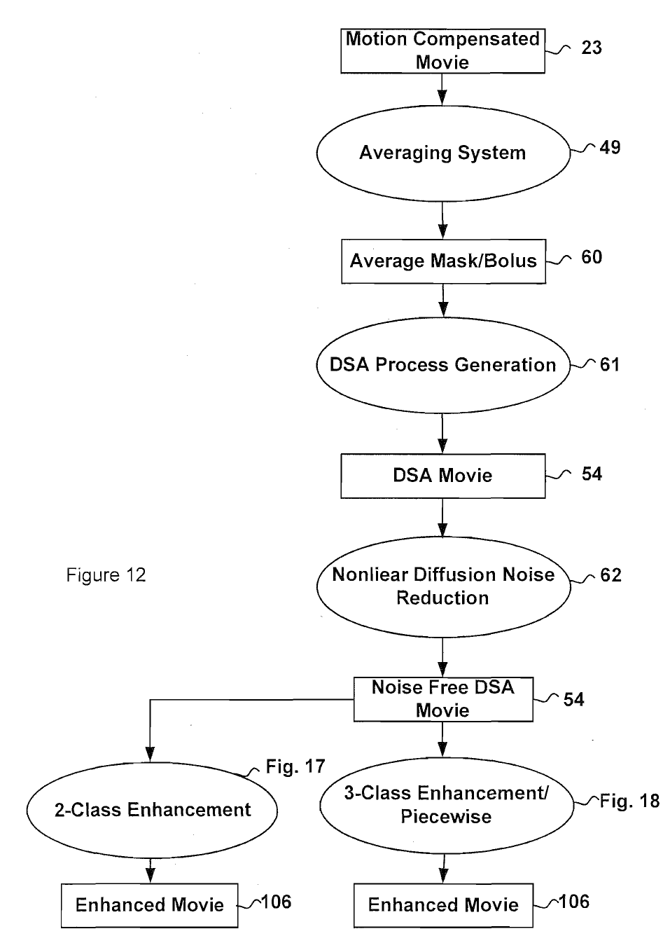

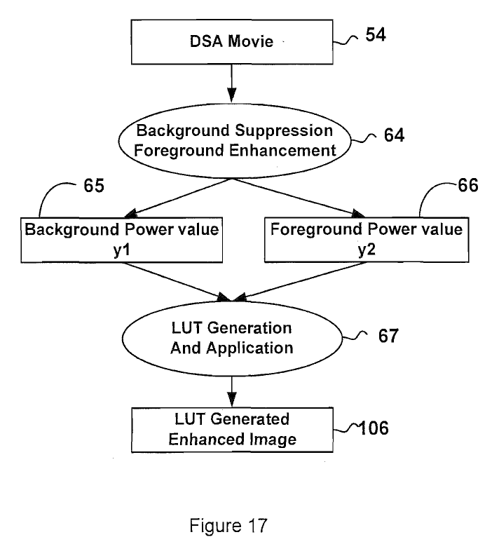

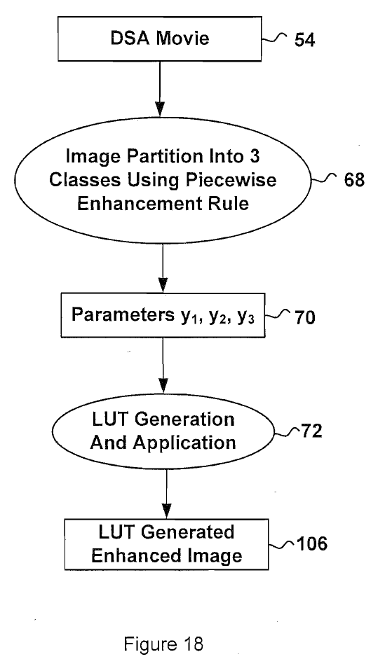

FIG. 12 illustrates the utilization of the motion corrected frames (23) to generate an enhanced DSA display or movie (106) (e.g., step 24 of FIG. 5). As shown a set of bolus frames and a set of mask frames are averaged together by an averaging system (49) to reduce the noise and slight registration errors. The average mask and average bolus frames (60) may still contain motion artifacts, since the frames were farther apart. The average images are registered together to remove this motion artifact. We obtain the subtraction image by computing a difference between pixel intensities of the mask image and the registered bolus image in a DSA process generation step (61). This is still a noisy image and we use noise removal processes (63) to reduce the noise. We call the noise removed image as the DSA image/movie (54). The intensities of the DSA image are normalized using method 1 (FIG. 17) (non-linear normalization) or method 2 (FIG. 18) (piece-wise non-linear intensity normalization) depending upon the average gray value of the image as well as histogram distribution. In either case, an enhanced movie is generated for display 106. DSA Generation System

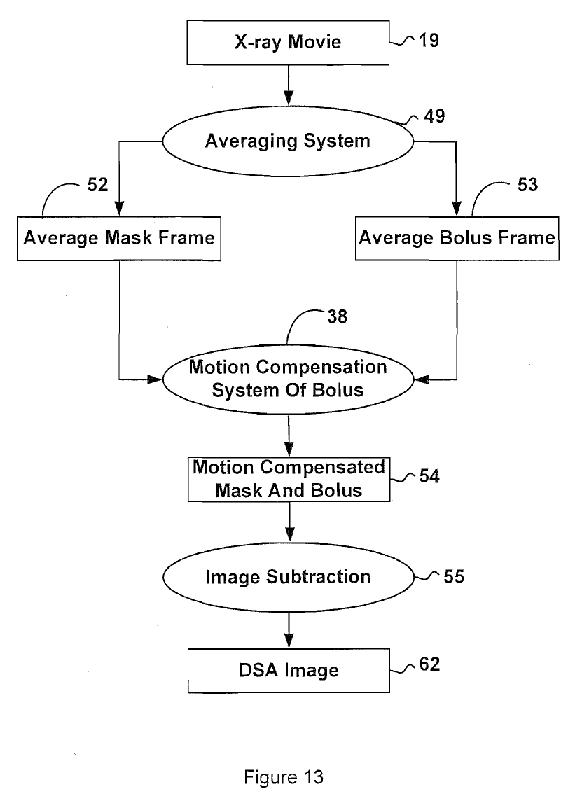

The DSA process generation (61) utilizes a set of mask frames (e.g., 4 mask frames) and set of bolus frames (e.g., four bolus frames) are used to generate the DSA image. See FIG. 13. The four mask frames and four bolus frames are aligned among themselves, respectively, as a consequence of mask frame identification. These images are averaged together to generate average mask image and average bolus image using the following averaging method (51):

### Mask Averaging

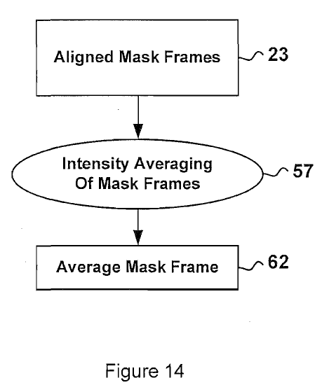

The four frames extracted as the mask images are used to create an average mask image (FIG. 14). The average is created by taking a pixel-wise averaging of the intensities of the 4 images. Let Fᵢ(x) represent intensity of image Fᵢ at pixel location x, where x is a 2-dimensional position vector corresponding to row and column number of the pixel x. Then, the average mask image (52) is computed as:

$${M}_{ave} ( x ) = \frac{{F}_{n - 4} ( x ) + {F}_{n - 3} ( x ) + {F}_{n - 2} ( x ) + {F}_{n - 1} ( x )}{4} , x ∈ Ω \qquad ( 3 )$$

where, Mₐᵥₑ represents the average mask image, Ω represents the image domain and frame no. Fₙ corresponds to the first bolus image.

Since the 4 frames are already aligned together through registration in the mask selection process, they are in same co-ordinate system. In other words, the images do not have differences due to motion and all background structures lie on top of one another. An average over already aligned structures reduces the noise in the images and increases the signal-to-noise ratio. In contrast to un-registered images, the averaging does not cause blurring of images and produces a sharp image with reduced noise.

### Bolus Averaging

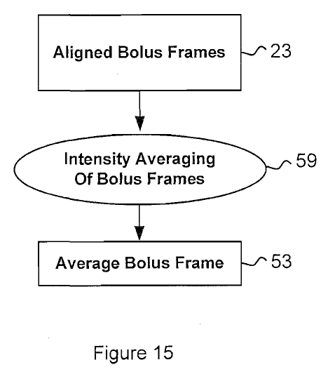

The 4 frames with dye are used to create an average bolus image (FIG. 15). The average (53) is created by taking a pixel-wise averaging of the intensities of the 4 images (59). Let Fᵢ(x) represent intensity of image Fᵢ at pixel location x, where x is a 2-dimensional position vector corresponding to row and column number of the pixel x. Then, the average bolus image is computed as:

$${B}_{ave} ( x ) = \frac{{F}_{n} ( x ) + {F}_{n + 1} ( x ) + {F}_{n + 2} ( x ) + {F}_{n + 3} ( x )}{4} , x ∈ Ω \qquad ( 4 )$$

where, Bₐᵥₑ represents the average bolus image, Ω represents the image domain and frame no. Fₙ corresponds to the first bolus image.

The frames are already aligned together through registration in the bolus selection process and are in same co-ordinate system (23). An average over already aligned structures reduces the noise in the images and increases the signal-to-noise ratio. In contrast to un-registered images, the averaging does not cause blurring of images and produces a sharp image with reduced noise.

### Computing DSA Images(61)

Digital subtraction Angiography (DSA) is used to extract the enhanced blood vessels using a contrast enhancing agent injected into the blood stream. This involves computing pixel-wise subtraction of bolus image from the mask image. However, images (52, 53) have to be motion-corrected before the above difference is calculated. For doing this, average mask and average bolus images are registered together (38). Let Mₐᵥₑ′ represent the average mask aligned with average bolus image Bₐᵥₑ through registration (54). The DSA image is computed by subtracting (55) the intensity values of average bolus image from the intensity values of registered average mask image at each pixel location, i.e. if the intensity of DSA is represented as the image at pixel x as I(x), then, I(x)=M′ₐᵥₑ(x)−Bₐᵥₑ(x)xεΩ, where Ω represents the image domain. This module provides a DSA movie as its output (56).

### Intensity Normalization

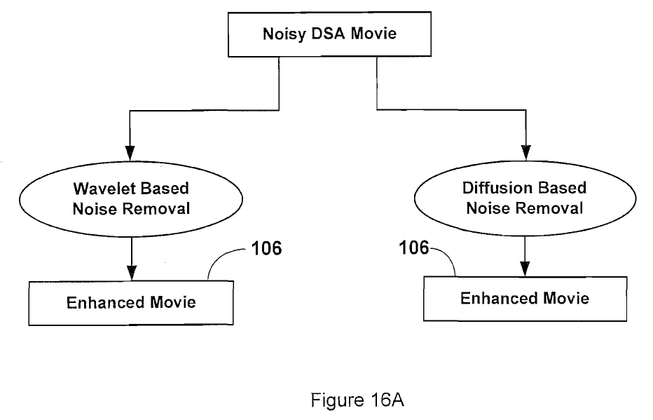

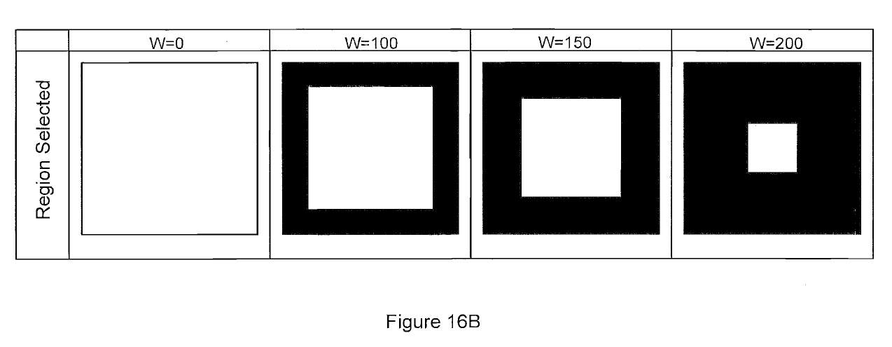

Depending upon the original intensity distribution of the images, two different methods are utilized to normalize the intensities of the images to enhance the contrast between the dye and the background. The main idea here is to reduce the intensities of dye and to increase the intensity values of the background, as dye appears darker and background appears brighter in the subtraction images. Some images have low intensity range in the dye and the contrast is enhanced using a non-linear method to further enhance this contrast. The following steps are performed for the same:
1. Linear Normalization of the images (FIG. 17): The difference images may contain positive and negative values, which needs to be resealed to values from 0 to 255. This id done by linear normalization of intensities using the maximum and minimum value of intensities in the subtraction images. Let I₁ and I₂ represent the lowest intensity value and highest intensity value, respectively, in the subtraction image. Then the image intensity is normalized using the following linear rule:

$${I}_{new} ( x ) = 255 \frac{{I}_{old} ( x ) - {I}_{1}}{{I}_{2} - {I}_{1}} \qquad ( 5 )$$

where, I_{old}(x) represents the original intensity value at pixel location x, and I_{new}(x) represents the new intensity value assigned to that location. Edge based linear normalization: The overall intensity of the image is regulated by the total x-ray dose, and the contrast between the background structures and the blood vessels is determined by the contrast enhancing dye. The field of view (FOV) is chosen such that the region of interest, i.e. blood vessels are in the middle of the images. To enhance the relative contrast of the image, more emphasis should be given to the region in the interior of images than the region closer to the edges. An image edge based normalization technique is utilized, in which a band of pixels close to the edges is removed and the maximum and minimum values are computed inside the inner rectangle as shown in FIG. 16B. The figure shows that while increasing width to a certain extent improves the contrast, a large width of band causes the region of consideration to be very small resulting in an over-sensitive system, as can be seen from the last image in the figure. Since an optimum size for the window varies from an image to next, a method is provided for computing width based on the signal-to-noise ratio. The width yielding best signal-to-noise ratio will be used as the optimum width for minimum/maximum calculations for linear normalization of the intensities.
2. Non-Linear Normalization of the images: The linearly normalized images only scale intensities to be in the range of 0-255. To increase the contrast between the dye and the background, non-linear resealing is needed. Two rules are provided for contrast enhancement of the images:
   a. 2-Class Enhancement (FIG. 17): This method works best for the images where the intensity range of dye lies in lower half of the intensity ranges. The following equation is used to re-assign intensity values at a location x (67):

$${I}_{new} ( x ) = \{ \begin{matrix}127 {( \frac{{I}_{old} ( x )}{127} )}^{{y}_{1}} , & {I}_{old} ( x ) ∈ [ 0 \text{,} 127 ] \\ 128 + 128 {( \frac{{I}_{old} ( x ) - 128}{128} )}^{{y}_{2}} , & {I}_{old} ( x ) ∈ [ 128 \text{,} 255 ]\end{matrix} \qquad ( 6 )$$

For contrast enhancement, y₁ is chosen to be greater than 1.0 and y₂ is chosen to be less than 1.0.
b. Piece-wise non-linear normalization (FIG. 18): The non-linear method described in part (a) above does not work well if the dye intensities cross the threshold value of 128. In some images, the intensity value at dye reaches upto 160, and the mean intensity value of image is around 180. In such cases, the non-linear method tends to lighten the already light regions of dye. In these cases, an alternative function using three different rules for three different classes of image intensities (68) is used to map the intensity values, described by the following equation:

$${I}_{new} ( x ) = \{ \begin{matrix}{{I}_{1} ( \frac{{I}_{old} ( x )}{{I}_{1}} )}^{{y}_{1}} , & {I}_{old} ( x ) ∈ [ 0 , {I}_{1} ] \\ {I}_{1} + ( {I}_{2} - {I}_{1} ) {( \frac{{I}_{old} ( x ) - {I}_{1}}{{I}_{2} - {I}_{1}} )}^{{y}_{2}} , & {I}_{old} ( x ) ∈ ( {I}_{1} , {I}_{2} ) \\ {I}_{2} + ( 255 - {I}_{2} ) {( \frac{{I}_{old} ( x ) - {I}_{2}}{255 - {I}_{2}} )}^{{y}_{3}} , & {I}_{old} ( x ) ∈ [ {I}_{2} , 255 ]\end{matrix} \qquad ( 7 )$$

where, 0≦I₁≦I₂≦255 and the range [I₁, I₂] represents a band that provides a smoother transition of mapping function. The value of the bands and the powers y₁, y₂ and y₃ (70) will be derived from the histogram (72) of intensity values of the subtraction image.

### Noise Reduction

In general, the images need to be de-noised for improving the quality of images before enhancement. The noise may be present in the form of salt-and-pepper noise in the images, and any intensity normalization may also cause the dots in the image background appear more prominent. It is therefore, desirable to remove the noise from the background before performing intensity normalization. Two methods are presented for removing noise from the DSA images.: wavelet smoothing and nonlinear diffusion (FIG. 16A). The methods are discussed below:
1. Wavelet based noise reduction: The wavelet based noise reduction strategy removes the noise from the background, while enhancing the blood vessels. Wavelet transforms are useful multi-resolution analysis tools in image processing and computer vision. The orthogonal wavelet transform of a signal f can be formulated by

$$f ( t ) = {\sum }_{k ∈ z} {c}_{J} ( k ) {ϕ}_{J , k} ( t ) + {\sum }_{J = 1}^{J} {\sum }_{k ∈ Z} {d}_{j} ( k ) {ϕ}_{j , k} ( t ) \qquad ( 8 )$$

where the cⱼ(k) is the expansion coefficients and the dⱼ(k) is the wavelet coefficients. The basis function φ_{j,k}(t) can be presented as

φ_{j,k}(t)=2^{-ji2}φ(2^{-j}t−k), (9)

where k, j are translation and dilation of a wavelet function φ(t). Therefore, wavelet transforms can provide a smooth approximation off(t) at scale J and a wavelet decomposition at per scales. For 2-D images, orthogonal wavelet transforms will decompose the original image into 4 different subband (LL, LH, HL and HH). The LL subband image is the smooth approximation of the original image. In our down sampling procedure, the first scale LL subband image, which has half size of the original one, will be applied as the down sampled image. The smoothing removes the noise from the image and provides a smoother and visually more appealing image, while providing a better signal-to-noise ratio.
2. Nonlinear diffusion based noise reduction: The second method to remove noise from background while enhancing the blood vessels is based on nonlinear diffusion. The nonlinear diffusion technique is based on partial differential equation (PDE) for noise smoothing. Given an image i(x,y,t) at time scale t, the diffusion equation is showed as follows:

$$\frac{\partial }{\partial t} I ( x , y , t ) = div ( c ( x , y , t ) ∇ I \qquad ( 10 )$$

where ∇ is the gradient operator, div is the divergence operator, and c(x, y, t) is the diffusion coefficient at location (x,y) at time t. With applying the divergence operator, the Eq. (4) can be rewritten as

$$\frac{\partial }{\partial t} I ( x , y , t ) = c ( x , y , t ) ∇ I + ∇ c ( x , y , t ) ∇ I \qquad ( 11 )$$

where Δ is the Laplacian operator. The diffusion coefficient c(x,y,t) is the key in the smoothing process and it should encourage homogenous-region smoothing and inhibit the smoothing across the boundaries. It is chosen as a function of the magnitude of the gradient of the brightness function, i.e.

c(x, y, t)=g(∥∇I(x, y, t)∥) (12)

The suggested functions for g(·) are the following two:

$$g ( ∇ I ) = {e}^{- {( \frac{‖ ∇ I ‖}{K} )}^{2}} \\ and \\ g ( ∇ I ) = \frac{1}{1 + {( \frac{‖ ∇ I ‖}{K} )}^{2}} \qquad ( 13 )$$

where K is the diffusion constant which controls the edge magnitude threshold. Generally speaking, a larger K produces a smoother result in a homogenous region than a smaller one. Here we apply diffusion technique on the input DSA images to smooth background and reduce noises.

### Overview

The series of images are acquired at different time instants and define a movie with a series of frames before, during and after the dye injection. The frames are therefore, available for original image mask and with contrast-enhancing dye injection. It is important to detect the frames before and after dye injection automatically to make it a feasible real-time system. One approach is to find intensity differences between successive frames, such that a large intensity difference is detected between the first frame after dye has reached the field of view (FOV) and the frame acquired before it. However, the patient may undergo some motion during the image acquisition causing such an intensity difference between even successive mask images. To avoid this, successive frames are aligned together, such that the motion artifacts are minimized. The subtraction image obtained after this will contain a near-zero value everywhere if both images belong to background. The first image acquired after the dye has reached the FOV will therefore cause a high intensity difference with the previous frame not containing the dye in FOV. The previous four registered frames are then collected as the mask frames, and the consecutive four frames with dye in FOV are extracted as the bolus frames.

The four bolus frames and four mask frames are averaged together to reduce the noise and slight registration errors. The average mask and average bolus frames may still contain motion artifacts, since the frames were farther apart. The average images are registered together to remove this motion artifact. A subtraction image may be obtained by computing a difference between pixel intensities of the mask image and the registered bolus image. The image at this point may be normalized and/or enhanced to provide a real-time output that may be utilized to, for example, guide a medical instrument in an interventional procedure.

The disclosed systems and methods provide numerous advantages including and without limitation fast and automatic detection of mask and bolus frames to be used for averaging as opposed to frames being are selected manually. Blurring effects in average image due to patient motion during the frame acquisition are reduced as all the frames are motion-corrected using image registration. As a result, the averages are sharp and do not contain artifacts due to patient's movements during the scan. The average structural image and the average image with injected dye are registered together and motion artifacts between the two images are minimized. This leads to minimizing the background structures showing up in the difference images, as can be seen in the results section before and after registration. Registration aligns the background structures and thus, the difference images contain much lesser unnecessary structures than the original un-registered images. The edge based normalization produces an output that ignores peaks and minimums of intensities occurring near the edge of the images, as such structures are generally not desired. The non-linear and piecewise non-linear image enhancement increases the contrast between the blood vessels and the background. This results in much improved contrast and very crisp subtraction images, in which the regions of interest are easily identifiable. The wavelet based noise reduction reduces the noise in background while enhancing the blood vessels thus improving the quality of output DSA image. The diffusion based noise reduction reduces the noise from the background resulting in improvement in image quality. The entire method may be automatic and streamlined as one single process with no human interaction, which makes it a superior method than the currently available methods, which require human interference at a number of steps. Results utilizing the above noted systems and methods are provided in Appendix A.

Any other combination of all the techniques discussed herein is also possible. The foregoing description has been presented for purposes of illustration and description. Furthermore, the description is not intended to limit the invention to the form disclosed herein. While a number of exemplary aspects and embodiments have been discussed above, those of skill in the art will recognize certain variations, modifications, permutations, additions, and sub-combinations thereof. It is therefore intended that the following appended claims and claims hereafter introduced are interpreted to include all such variations, modifications, permutations, additions, and sub-combinations as are within their true spirit and scope.

1. A method for use in a real-time medical imaging system, comprising:
obtaining a plurality of successive images having a common field of view, said images being obtained during a contrast media injection procedure;
identifying a first set of said plurality of images that are free of contrast media in said field of view;
identifying a second set of said plurality of images having contrast media in said field of view; and
generating a differential image based on differences between a first composite image associated with said first set of images and a second composite image associated with said second set of images.

2. The method of claim 1, further comprising:
displaying said differential image on a user display.

3. The method of claim 2, further comprising:
guiding a medical instrument while monitoring said user display.

4. The method of claim 1, wherein said first and second sets of images are identified in an automated process.

5. The method of claim 4, wherein said automated process comprises:
computing intensity differences between temporally adjacent images; and
identifying an intensity difference between two temporally adjacent images indicative of contrast media being introduced into a subsequent of said two adjacent images.

6. The method of claim 5, wherein said two temporally adjacent images define a contrast media introduction reference time and wherein:
identifying said first set of images comprises selecting a predetermined number of successive images before said contrast media introduction reference time; and
identifying said second set of images comprises selecting a predetermined number of successive images after said contrast media introduction reference time.

7. The method of claim 5, wherein computing intensity differences further comprises:
motion correcting each image, wherein each motion corrected imaged is registered to its immediately preceding image.

8. The method of claim 7, wherein said first and second sets of images comprise first and second sets of motion corrected images.

9. The method of claim 1, wherein said first and second composite images comprise:
a first average image generated from said first set of images; and
a second average image generated from said second set of images.

10. The method of claim 7, wherein said first and second sets of images are motion corrected prior to generating said first and second average images.

11. The method of claim 1, wherein generating a differential image comprises:
motion correcting said first and second composite images, wherein said first and second composite images are registered together.

12. The method of claim 11, wherein said composite images are registered together via an inverse consistent registration method.

13. The method of claim 12, wherein said inverse consistent registration method is computed using a B-spline parameterization.

14. The method of claim 11, wherein said differential image is generated by subtracting intensity values of one of said first and second composite images from the other of said first and second composite images.

15. The method of claim 14, wherein subtracting is performed at each pixel location of said composite images.

16. The method of claim 14, further comprising:
enhancing the contrast between the contrast media as represented in said differential image and background information of said differential image.

17. The method of claim 16, wherein enhancing the contrast comprises performing a linear normalization to rescale pixel intensities of said differential image.

18. The method of claim 17, wherein said linear normalization is performed based on the minimum intensity value and the maximum intensity value of said differential image.

19. The method of claim 18, further comprising:
selecting a region of interest from said field of view of said differential image, wherein said linear normalization is performed based on minimum and maximum intensity values in said region of interest.

20. The method of claim 16, wherein enhancing the contrast comprises performing a nonlinear normalization to rescale pixel intensities of said differential image.

21. The method of claim 20, wherein said nonlinear normalization is performed in first and second pixel intensity bands.

22. The method of claim 21, wherein said nonlinear normalization is performed in at least three pixel intensity bands.

23. The method of claim 16, further comprising performing a noise reduction process to remove noise from said differential image.

24. The method of claim 23, wherein said noise reduction process comprises at least one of:
a wavelet based noise reduction process; and
a nonlinear diffusion based noise reduction process.

25. A method for use in a real-time medical imaging system, comprising:
obtaining a plurality of successive images having a common field of view, said images being obtained during a contrast media injection procedure;
registering each of said plurality of images with a temporally adjacent image to generate registered images;
comparing intensities of temporally adjacent registered images for identifying a first image where contrast media is visible.

26. The method of claim 25, wherein identifying comprises identifying an intensity difference between adjacent images that is greater than a predetermined threshold.

27. The method of claim 25, further comprising:
selecting a first set of registered images temporally prior to said first image where contrast media is visible, wherein said first set of registered images define a mask set;
selecting a second set of registered images temporally subsequent to said first image where contrast media is visible, wherein said second set of register images define a bolus set.

28. The method of claim 27, further comprising;
generating a mask average image and a bolus average image; and
subtracting said bolus average image from said mask average image to generate a differential image.

29. The method of claim 28, further comprising:
reducing noise in said differential image; and
enhancing the contrast of said differential image.

30. A method for use in a real-time medical imaging system, comprising:
obtaining a plurality of successive images having a common field of view, said images being obtained during a contrast media injection procedure;
registering each of said plurality of images with a temporally adjacent image to generate a plurality of registered images;
averaging a mask set of registered images free of contrast media in said common field of view, wherein averaging generates an average mask image;
averaging a bolus set of registered images showing said contrast media in said common field of view, wherein averaging generates an average bolus image;
generating a differential image based on differences between said average mask image and said average bolus image;
removing noise from said differential image; and
enhancing contrast between pixels in said differential image.

31. The method of claim 30, further comprising:
registering said average mask image and said average bolus image prior to generating said differential image.
